(19) United States
(12) Patent Application Publication
(10) Pub. No.: US 2010/0331896 A1
(43) Pub. Date: Dec. 30, 2010

(54) Device for Anchoring a Tissue in a Bone

(76) Inventors: Régis Le Couedic, Bordeaux (FR); Denis Pasquet, Quinsac (FR)

Correspondence Address: BRINKS HOFER GILSON & LIONE, P.O. BOX 10395, CHICAGO, IL, 60610, US

(21) Appl. No.: 12/809,520

(22) PCT Filed: Dec. 22, 2008
(86) PCT No.: PCT/FR2008/001814

§ 371 Date: Jul. 21, 2010

(30) Foreign Application Priority Data
Dec. 21, 2007 (FR) 0709090

(51) Int. Cl. A61B 17/86

(52) U.S. Cl. 606/305

(57) ABSTRACT

It is a device (1) for anchoring a tissue (2) in a bone (3), which comprises an anchor (6) extended around an axis (7), comprising a head (8) pierced with an eyelet (24) for suture threads (5) to pass through, and a body (9) for inserting and maintaining the head in the bone. The body (9) has an external thread (11) for screwing into the bone. The anchor (6) comprises means (10, 10′) for fastening the head (8) onto the body (9), which means are arranged so as to fasten one to the other in the axial direction and to allow free rotation of one with respect to that other about said axis (7), such that the head can remain immobile when the body is screwed into the bone.

The present invention relates to a device for anchoring a tissue in a bone comprising an anchor extended around an axis comprising a head pierced with an eyelet for the passage of suture threads and a body for the insertion and retention of the head inside the bone.

It also relates to a method for attaching and retaining a tissue on a block.

The invention finds an application that is particularly important, although not exclusive, in the field of implants or anchors called second-rank anchors for, repairing the rotator cuff.

The rotator cuff is a set of tendons which essentially comprises four tendons used to attach the infraspinatus, supraspinatus, subscapularis and teres minor muscles on the head of the humerus.

Devices making it possible to attach a flexible tissue that has been torn off to the corresponding bone are already known.

In particular, devices such as sutures, screws, anchors or pins have been used in the prior art to allow such a reattachment.

An implant commonly consisting of a small anchor to be impacted, free to slide over fifteen or so millimeters in the distal portion of the prehensile instrument and which is premounted on said instrument and for single use, is therefore known.

At the end of the gripper, a small eyelet is assembled which serves amongst other things as an abutment to the anchor.

This type of device is routinely implanted by arthroscopy, that is to say by inserting the prehensile instrument through a mini-incision with or without video assistance.

More precisely, the process used is as follows: the process begins with the attachment of the first-rank anchors which are put in place in a manner known per se, the strands originating from these anchors being passed through the tissue to be attached, then through the eyelet of a second-rank anchor implanted outside the tissue. To do this, the latter is lowered into a hole previously made with a punch, in a lateral position relative to the first-rank anchor.

The strands then being held tight in the eyelet, the anchor is then impacted and will thereby secure the strands which will then be cut level with the anchor.

Such a device has drawbacks.

In particular, it has a mediocre mechanical resistance to being torn out relative to a screwed anchor and requires guidance during impacting.

Also known (WO 01/54586) is a device for anchoring suture thread in a tissue comprising an end point that does not require prior drilling.

The device is furnished with locking branches which spread when the drilling has been done with said point which is forcibly inserted into the tissue, which has the drawback of being complicated and requires means for controlling the spreading.

Also known (US 2006/0259076) is a suture chain immobilized at the bottom of a screwed implant.

The latter however allows neither tightening by tensioning the chain immobilized by its links nor fine adjustment.

The object of the present invention is to propose a second-rank anchor which, better than those previously known, meets the requirements of the practice, notably in that it proposes an implant that has an excellent hold, is easy to apply and is simple, which allows an excellent tightening of the suture threads, is adjustable and can be fine-tuned during and after screwing, is not very costly and requires no particular additional ancillary element.

To do this, it is based on the idea of forming an implant that is also screwed like that of the first rank, but in two portions, namely one portion constituting the body of the anchor and the other forming the eyelet in which the suture strands will be passed.

These two portions are assembled in a captive manner during the manufacture of the device, while allowing the screwing of the body of the anchor while keeping the eyelet receiving the strands immobile in the bone.

Because of the slow and precise method of insertion that screwing represents, it is (now) no longer necessary to have an ancillary element serving as a guide axis as in the prior art.

This therefore makes it possible to dispense with the premounted, single-use insertion instrument, which represents a considerable cost saving.

Moreover, the eyelet is designed with a diameter close or equal to that of the core of the screwed portion, and therefore itself serves as a guide support.

For this purpose, the invention essentially proposes a device for anchoring a tissue in a bone comprising an anchor, extended around an axis, comprising a head forming a distal portion pierced with an eyelet for the passage of suture threads and a body for the insertion and retention of the head inside the bone, characterized in that the body is furnished with an outer thread for screwing into the bone, and in that said anchor comprises means for attaching the head to the body, said means being arranged to attach them together in the axial direction and to allow the free rotation of one relative to the other about said axis so that the head can remain immobile when the body is screwed into the bone.

“Head” must therefore in this instance be understood to be the end, distal, portion of the anchor which can be separated from and is free to rotate relative to the body of the anchor, which is screwed.

In advantageous embodiments, one and/or the other of the following arrangements is/are used:
the body and the head are cylindrical or substantially cylindrical, of the same diameter or substantially of the same diameter;
the means for attachment between the head and the body are clipping means;
the clipping means comprise a female portion and a male portion, the female portion being formed of two opposite jaws like portions of a cylinder furnished with a cylindrical central recess, separated by a transverse slot allowing them to be moved apart elastically and comprising an end portion forming a counterbore for retention of a protrusion secured to the male portion once the latter has been inserted by force into said female portion, said male portion being of cylindrical shape matching that of the recess and the male and female portions comprising, at their ends, contact surfaces arranged in order to act together in gentle friction against one another once the clipping has been achieved;
the clipping means comprise a female portion and a male portion, the female portion being formed by a doubly cylindrical axial recess forming an internal indentation for retaining one end as a bipartite protrusion of said male portion, the latter comprising two semicylindrical branches separated from one another by a central slot allowing them to move closer together for clipping of said protrusion into the corresponding recess and the male and female portions comprising, at their ends, contact surfaces arranged in order to act together in gentle friction against one another once the clipping has been achieved;
the male portion is secured to the body and the female portion is secured to the head;
the anchor is made of titanium and/or polyether ether ketone (PEEK) and/or of bioresorptive material;
the device comprises at least two anchors suitable for being attached directly in the outer portion of the head of the humerus at the anatomical collar, then called second-rank anchors;
the device also comprises at least one set of first-rank anchors suitable for being attached in the bone in order to be placed subsequently beneath the tissue and suture threads secured to said set that are suitable for piercing the tissue, said threads passing through the eyelets of the second-rank anchors in order to form a network of threads for flattening the tissue on the head of the humerus.

The invention also proposes a method for attaching and holding a tissue on a block, for example for the attachment of a tissue of the tendon or muscle type on a head of a humerus, characterized in that a set of first-rank anchors furnished with attachment threads is attached in the block, the strands of the attachment threads originating from this set are passed through the tissue and then through the eyelets of second-rank anchors and then the second-rank anchors are inserted by screwing into respective holes previously produced by a punch in the outer portion of the block, for example the head of the humerus, at the anatomical collar, the heads or ends furnished with these eyelets being directed toward the block and held without turning during the screwing of the anchor, the strands being held in tension until the screwing is complete.

The present invention will be better understood on reading the following description of an embodiment given below as a nonlimiting example.

The description refers to the drawings accompanying it in which:

FIG. 1 is a view in perspective of a rotator cuff attached to the corresponding bone via a first set of first-rank anchors and a second set of second-rank anchors according to the invention.

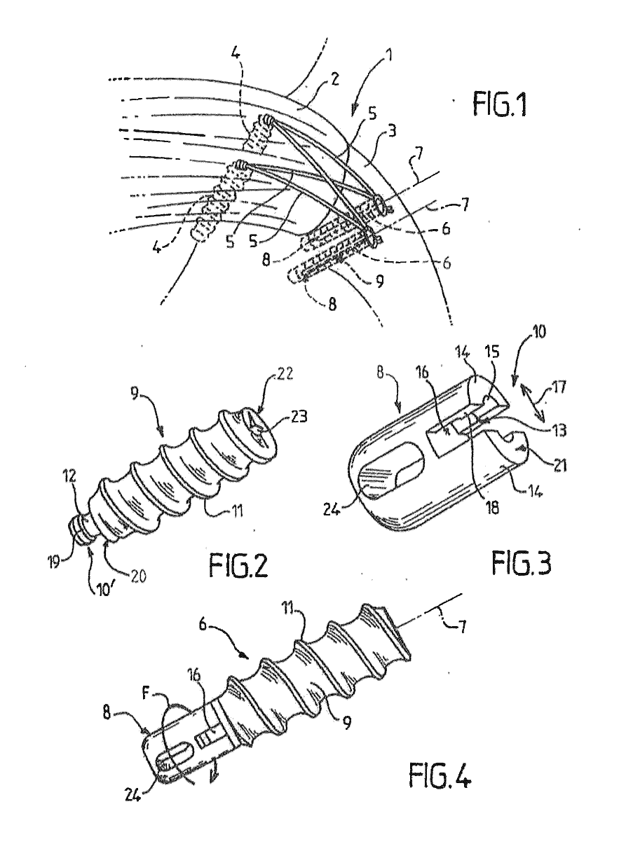

FIG. 2 is a view in perspective of one embodiment according to the invention of the body for inserting and holding the head inside the bone.

FIG. 3 is a view in perspective of a rotatable head pierced with an eyelet for the passage of a suture thread, belonging to an anchor according to the invention.

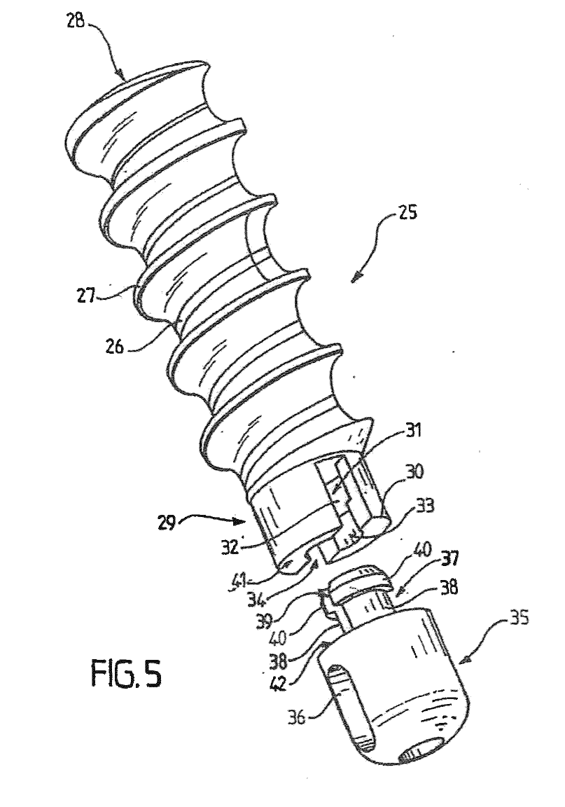

FIG. 4 shows the elements of FIGS. 2 and 3 assembled to form the anchoring device according to the embodiment of the invention that is more particularly described here.

FIG. 5 shows in perspective another embodiment of the device according to the invention before the head is clipped onto the body.

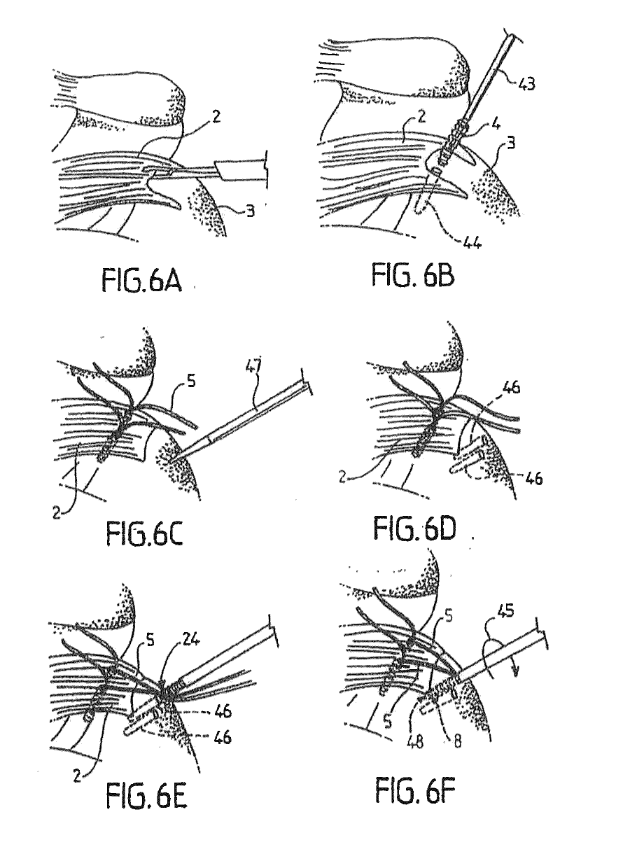

FIGS. 6A to 6F show schematically in perspective the method for putting in place the devices for anchoring a tissue according to the embodiment of the invention that is more particularly described here.

FIG. 1 shows a device 1 for anchoring a tissue 2 in a bone 3 comprising a set of two anchors 4, called first-rank anchors, furnished with suture threads 5 originating from this set through the tissue 2 and two anchors 6, called second-rank anchors, extended around axes 7.

The anchors 6 each comprise a head 8 that is pierced for the passage of the suture threads 5 and a body 9 for the insertion and holding of the head 8 inside the bone 3.

With reference to FIGS. 2, 3 and 4, the second-rank anchor 6 comprises means 10, 10′, for attaching the head 8 to the body 9, which will be explained in detail below and which are arranged in order to attach them to one another in an axial direction and to allow one to rotate relative to the other about the axis 7 (arrow F).

The body 9 being furnished with an outer thread 11 for screwing into the bone, the rotational separation allows the head to remain immobile when the body is screwed into the bone.

In the embodiment more particularly described, the attachment means 10, 10′ are means for clipping a male portion 12 into a female portion 13.

More precisely, the female portion is formed of two opposite jaws 14 like portions of a cylinder furnished with a cylindrical central recess 15, separated by a transverse slot 16 allowing them to be moved apart elastically (arrow 17) and comprising an end portion 18 forming a counterbore for retention of a protrusion 19 secured to the male portion 12 once the latter has been inserted by force into said female portion, said male portion 12 being of cylindrical shape matching that of the recess 15 and the male and female portions comprising, at their ends, flat contact surfaces 20, 21 arranged in order to act together in gentle friction against one another once the clipping has been achieved (see FIG. 4).

In this more particular embodiment, the male portion is secured to the threaded body and the female portion secured to the head.

In the embodiment more particularly described, the body and the head are substantially cylindrical, made of titanium, of PolyEther Ether Ketone or PEEK, or of a plastic material formed from polylactide acid (a bioresorptive material).

The body has for example a length of 2 cm, an inner diameter of 4 mm and comprises a screw pitch of 1.5 to 3 mm. In the outer upper portion 22 of the cylinder, it is furnished with a slot 23 for driving action by a screwdriver.

The head 8, for its part, is furnished with a central eyelet 24, for example of oval section, for suture strands to pass right through.

FIG. 5 shows a device 25 comprising a cylindrical body 26 furnished with an outer thread 27 known per se and with a portion 28 for actuation by a screwdriver (not shown). The body comprises at its end 29 a female portion 30 formed of an axial recess 31 comprising two portions of axial cylinders 32 and 33 the innermost of which forms an inner indentation.

In this embodiment, the two cylinder portions are for example split by a transverse slot 34 over the whole height of the cylinders.

The device 25 also comprises a head 35 furnished with an eyelet 36 passing right through the head, and, at its end for connection with the body, a male portion 37 comprising two semicylindrical branches 38 separated from one another by a central slot 39 allowing them to be brought together by clipping their end in the form of cylindrical or beveled half-protrusions 40.

The male and female portions comprise at their respective ends surfaces 41, 42 for interaction with gentle friction.

As already indicated above, shown in FIG. 1 is a complete device according to one embodiment of the invention with first-rank anchors of the known type, obtained by screwing into the bone previously prepared by impact.

More precisely, and with reference to FIGS. 6A to 6F, the method of attachment with the aid of a device according to the invention will now be described.

In the rest of the description, the same reference numbers will be used to designate the same elements.

This therefore involves attaching and holding the tissue 2 on the head of the humerus 3 with the aid of the device 1.

First of all a first-rank set of anchors 4 is attached furnished with attachment threads (not shown in FIG. 6B because they are inside the screwing tool 43).

To do this, after having made the holes 44 in the bone, for example with a punch, the first-rank implants 4 are screwed in a manner known per se.

The strands 5 of the attachment threads originating from this set are then passed through the tissue 2 and then through the eyelets 24 of the second-rank anchors 6 (see FIG. 6E).

The second-rank anchors 6 are then screwed (arrow 45) into respective holes 46 previously made by a punch 47 in the bone 3. The heads 8 furnished with these eyelets are directed toward the inside of the bone and held without turning during the screwing, by virtue of the possibilities of rotation that exist between the head and the body.

The head furnished with an eyelet is held by friction coupling of the eyelet furnished with the strand 5 in the lower portion 48 of the implant sheath. The tightening of the strands 5, in order to finally attach and hold the tissue as shown in FIG. 1, is obtained during the screwing of the anchor and by action on the outer strands which slide through the central cat or eyelet 24.

As goes without saying and as also results from the foregoing, the present invention is not limited to the embodiments more particularly described. In contrast, it covers all the variants thereof and in particular those in which a single second-rank anchoring device is put in place and/or the male portion is secured to the eyelet instead of being secured to the body.

For its part, the attachment method also applies in fields other than that of surgery.

1. A device (1) for anchoring a tissue (2) in a bone (3) comprising an anchor (6), extended around an axis (7), comprising a head forming a distal portion (8) pierced with an eyelet (24) for the passage of suture threads (5) and a body (9) for the insertion and retention of the head inside the bone, characterized in that the body (9) is furnished with an outer thread (11) for screwing into the bone and in that said anchor (6) comprises means (10, 10′) for attaching the head (8) to the body (9), said means being arranged to attach them together in the axial direction and to allow the free rotation of one relative to the other about said axis (7) so that the head can remain immobile when the body is screwed into the bone.

2. The device as claimed in claim 1, wherein the body (9) and the head (8) are cylindrical or substantially cylindrical, of the same diameter or substantially of the same diameter.

3. The device of claim 1, wherein the means (10, 10′) for attachment between the head (8) and the body (9) are clipping means.

4. The device as claimed in claim 3, wherein the means (10, 10′) for clipping comprise a female portion (13) and a male portion (12), the female portion (13) being formed of two opposite jaws (14) like portions of a cylinder furnished with a cylindrical central recess (15), separated by a transverse slot (16) allowing them to be moved apart elastically and comprising an end portion (18) forming a counterbore for retention of a protrusion (19) secured to the male portion (12) once the latter has been inserted by force into said female portion, said male portion (12) being of cylindrical shape matching that of the recess and the male and female portions comprising, at their ends, contact surfaces (20, 21) arranged in order to act together in gentle friction against one another once the clipping has been achieved.

5. The device as claimed in claim 3, wherein the clipping means comprises a female portion (30) and a male portion (37), the female portion being formed by a doubly cylindrical axial recess (32, 33) forming an internal indentation for retaining one end as a bipartite protrusion (40) of said male portion, the latter comprising two semicylindrical branches (38) separated from one another by a central slot (39) allowing them to move closer together for clipping of said protrusion into the corresponding recess (32) and the male and female portions comprising, at their ends, contact surfaces (41, 42) arranged in order to act together in gentle friction against one another once the clipping has been achieved.

6. The device of claim 4, wherein the male portion (12) is secured to the body (9) and the female portion (13) is secured to the head.

7. The device of claim 1, wherein the anchor (1) is made of one or more of titanium, PEEK, or and/or of bioresorptive material.

8. An assembly for the anchoring of a tissue (2) in a bone (3), characterized in that it comprises at least two anchoring devices (1) of claim 1, forming anchors called second-rank anchors, at least one set of anchors (4), called first-rank anchors, and suture threads (5) secured to said set.

9. The device of claim 5, wherein the male portion (12) is secured to the body (9) and the female portion (13) is secured to the head.
